(19) United States
(12) Patent Application Publication
(10) Pub. No.: US 2017/0157108 A1
(43) Pub. Date: Jun. 8, 2017

(54) LIQUID FORMULATION COMPRISING MONTELUKAST OR PHARMACEUTICALLY ACCEPTABLE SALT THEREOF AND METHOD FOR PREPARING SAME

(71) Applicant: HANMI PHARM. CO., LTD., Hwaseong-si, Gyeonggi-do (KR)

(72) Inventors: Taek Kwan KWON, Suwon-si (KR); Xue Wen YAN, Shunyi District (CN); Zhao Zhen WANG, Shunyi District (CN); Yong Il KIM, Suwon-si (KR); Jae Hyun PARK, Suwon-si (KR); Jong Soo WOO, Suwon-si (KR)

(73) Assignee: HANMI PHARM. CO., LTD., Hwaseong-si, Gyeonggi-do (KR)

(21) Appl. No.: 15/039,089

(22) PCT Filed: Dec. 17, 2014
(86) PCT No.: PCT/KR2014/012485

§ 371 Date: May 25, 2016

(30) Foreign Application Priority Data
Dec. 19, 2013 (KR) 10-2013-0159636

(51) Int. Cl. A61K 31/47; A61K 47/22; A61K 47/32; A61K 47/14; A61K 47/40; A61K 47/44; A61K 9/08; A61K 47/26

(52) U.S. Cl. CPC A61K 31/47; A61K 9/08; A61K 47/22; A61K 47/26; A61K 47/14; A61K 47/40; A61K 47/44; A61K 47/32

(57) ABSTRACT

The present invention relates to a liquid formulation comprising a solubilizer and montelukast or pharmaceutically acceptable salt thereof in a solution state as an active ingredient, and a method for preparing same. The liquid formulation of the present invention allows montelukast or pharmaceutically acceptable salt thereof to be substantially dissolved and present in a solution state, thereby preventing decrease of dissolution over time and increasing its bioavailability. In addition, because the liquid formulation of the present invention shows superior stability, taste and flavor, its medication compliance can be enhanced, and it can be effectively utilized for asthma and allergic rhinitis patients.

### FIELD OF THE INVENTION

The present invention relates to a liquid formulation comprising montelukast or pharmaceutically acceptable salt thereof as a pharmacologically active ingredient, and a method for preparing same. More particularly, the liquid formulation contains montelukast or pharmaceutically acceptable salt thereof in a solution state by employing a solubilizer, resulting in increased bioavailability or improved stability and medication compliance.

### BACKGROUND OF THE INVENTION

Asthma is an inflammatory airway obstructive disease with symptoms of dyspnea and severe cough caused by swelling of bronchial mucosa due to bronchial inflammation, and bronchial obstruction due to bronchial muscle spasm. Patients with asthma show symptoms such as wheezing, dyspnea and cough etc. due to airway obstruction, and the symptoms have a tendency get worse at night than in daytime. Also, they may have difficulties in breathing due to excessive sputum production. In severe cases, asthmatic attacks may be life-threatening. Asthma is a problematic disease prevalent worldwide, and about 300 million asthma patients are estimated to be present. A half of childhood asthma cases persists until adulthood and continues as adult asthma. The prevalence of asthma among children and adults confirmed by a standard survey method amounts to 1 to 18%, albeit with differences among countries.

Therapeutic agents of asthma are mainly divided into disease-controlling agents and symptom-alleviating agents. The disease-controlling agents are used for controlling symptoms of asthma mainly by anti-inflammatory effects. The symptom-alleviating agents are medications to alleviate symptoms in a short time period by rapidly dilating airway, which are used when necessary.

Montelukast, a type of the disease-controlling agents, is an antagonist that inhibits cysteinyl leukotriene CysLT1 receptor, and has been used for the treatment and prevention of leukotriene-mediated diseases and disorders. Specifically, montelukast is known to be effective for allergic rhinitis, atopic dermatitis, chronic urticaria, sinusitis, nasal polyps, chronic obstructive pulmonary disease, conjunctivitis including rhino-conjunctivitis, migraine, cystic fibrosis, and viral bronchiolitis, etc. (S E Dahlen, Eur. J. Pharmacol., 533 (1-3), 40-56 (2006)). Also, it has a great advantage that it is effective for aspirin-sensitive asthma patients and has very few side effects. At present, Singulair™ (MSD), an agent employing sodium montelukast as its pharmacologically active ingredient, is approved for the treatment of asthma in adults and children over 6 months of age, and commercially available.

Regarding dosage regimens, doses and dosage forms, Singulair™ is commercially available with dosage forms of 10 mg tablets to be taken once a day by adults, 4 to 5 mg chewable tablets to be chewed and taken once a day by children aged 2 to 14 years, and 4 mg fine granules to be taken once a day by children aged 6 months to 2 years. Among them, the fine granules has a disadvantage that it should be taken directly p.o., or mixed in soft foods such as, for example, small amount (e.g. 5 mL or less) of baby food, mother's milk or porridge which is colder than room temperature and be taken immediately (i.e., within 15 minutes). Furthermore, being unstable to light, humidity and heat, montelukast produces degradation products such as montelukast sulfoxide (as shown in Formula 1 below) and montelukast cis-isomer (as shown in Formula 2 below), and so on when it is decomposed. According to a literature (M. M. Al Omani et al., J. Pharm. and Biomed. Anal., 45, 465-471 (2007)), it has been reported that upon exposure of Singulair™ chewable tablet to sunlight, a commercially available product employing sodium montelukast as its main ingredient, montelukast sulfoxide increases up to 2.4%, and upon exposure of montelukast to sodium, etc. for 6 hours in 0.1 M solution of hydrochloric acid, montelukast cis-isomer increases up to 14.6%. These results indicate that it is not easy to maintain stability of montelukast-containing products over time.

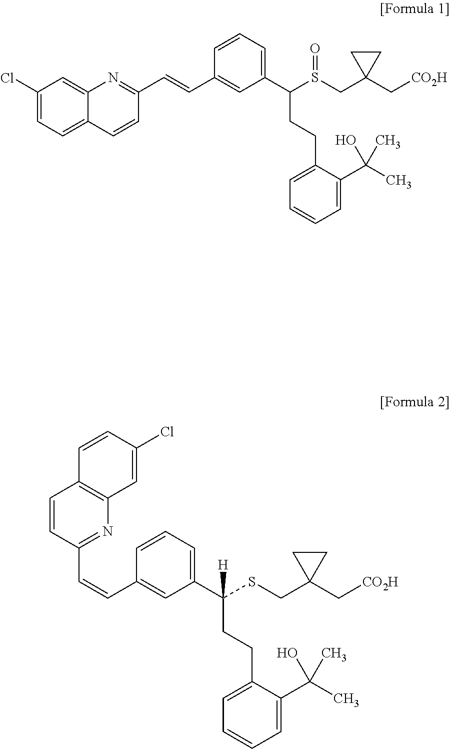 [SMILES: CC(C)(O)c1ccccc1CCC(c1cccc(/C=C/c2ccc3ccc(Cl)cc3n2)c1)S(=O)CC1(CC(=O)O)CC1 ; CC(C)(O)c1ccccc1CC[C@@H](SCC1(CC(=O)O)CC1)c1cccc(/C=C\c2ccc3ccc(Cl)cc3n2)c1]

Among the dosage forms of the therapeutic agents, oral liquid formulations, such as syrups, suspensions, elixirs, medicated spirits and liquid, have several advantages. They allow patients to control doses easily and can be conveniently taken by toddlers, children and the elderly patients. In comparison to solid formulations such as tablets, chewable tablets and granules, the bioavailability of the oral liquid formulations can be increased and individual variations can be decreased. Also, medication compliance of patients can be enhanced by improving taste and flavor by addition of sweeteners and flavoring agents.

Most of montelukasts or pharmaceutically acceptable salts thereof can be dissolved in purified water, but they become decomposed into other materials or produce precipitation by recrystallization over time, and the solubility thereof decreases. In addition, the pharmacologically active ingredient tends to decompose rapidly in an aqueous solution when dissolved in water, or related substances tend to increase when it is left in aqueous solution for a long time. Also, as montelukast or its pharmaceutically acceptable salt shows significantly lower solubility in an aqueous solution with high ionic strength (e.g., artificial gastric juice, artificial intestinal juice, etc.) than in purified water, there is a problem that its bioavailability is low in oral cavity or gastro-intestinal tract when actually taken by a patient. For these reasons, it is not easy to develop a liquid formulation employing montelukast or pharmaceutically acceptable salt thereof, and there is no product commercially available or developed yet.

### SUMMARY OF THE INVENTION

Therefore, it is an object of the present invention to provide a liquid formulation comprising montelukast or pharmaceutically acceptable salt thereof in a solution state as a pharmacologically active ingredient, with improved dissolution, stability and medication compliance.

It is a further object of the present invention to provide a method for preparing such a liquid formulation.

In accordance with one aspect of the present invention, there is provided a liquid formulation comprising: a solubilizer selected from the group consisting of cyclodextrin, polyvinyl caprolactam-polyvinyl acetate-polyethylene glycol graft copolymer, polyoxyethylene derivative and a water-soluble polymer; and montelukast or pharmaceutically acceptable salt thereof in a solution state as a pharmacologically active ingredient.

In accordance with another aspect of the present invention, there is provided a method for preparing a liquid formulation, comprising the steps of: preparing solution 1 by adding to purified water one or more pharmaceutically acceptable additives selected from the group consisting of a sweetening agent, a flavoring agent, a preservative, an emulsifying agent, a coloring agent and a stabilizer, and stirring resulting mixture; preparing solution 2 by adding to purified water a solubilizer selected from the group consisting of cyclodextrin, polyvinyl caprolactam-polyvinyl acetate-polyethylene glycol graft copolymer, polyoxyethylene derivative and a water-soluble polymer, and stirring resulting mixture, and then adding montelukast or pharmaceutically acceptable salt thereof to the mixture and stirring resulting mixture; and mixing solution 1 with solution 2, and adjusting the concentration of resulting solution by adding purified water thereto.

Because the liquid formulation of the present invention comprising montelukast or pharmaceutically acceptable salt thereof, allows its pharmacologically active ingredient to be present in a solution state, the decrease of dissolution over time can be prevented, patients can easily take the formulation owing to its favorable taste and flavor, and medication compliance can be enhanced by convenient dose control. In addition, it shows improvement in homogeneity of drug effect because recrystallization, precipitation or separation of layers does not take place even when stored for a long time, and can be effectively utilized for asthma and allergic rhinitis patients.

### BRIEF DESCRIPTION OF DRAWINGS

FIG. 1 illustrates the results of the montelukast dissolution test after the storage of the liquid formulations of Examples 1 to 5 at room temperature.

FIG. 2 illustrates the results of the montelukast dissolution test after the storage of the liquid formulations of Comparative Examples 1 to 8 at room temperature.

FIGS. 3 and 4 illustrate the amount of montelukast sulfoxide and total related substances of montelukast produced when liquid formulations of Examples 1 to 5 and Comparative Examples 1 to 8 were stored under accelerated conditions, respectively.

### DETAILED DESCRIPTION OF THE INVENTION

The present invention provides a liquid formulation containing: a solubilizer selected from the group consisting of cyclodextrin, polyvinyl caprolactam-polyvinyl acetate-polyethylene glycol graft copolymer, polyoxyethylene derivative and a water-soluble polymer; and montelukast or pharmaceutically acceptable salt thereof in a solution state as a pharmacologically active ingredient. The montelukast or pharmaceutically acceptable salt thereof of the present invention can be, for example, montelukast, montelukast sodium, montelukast potassium, montelukast strontium, montelukast calcium, montelukast magnesium or montelukast ammonium, among which montelukast sodium is preferred.

The montelukast or pharmaceutically acceptable salt thereof can be employed (as montelukast) in an amount of 0.005 to 10 weight %, preferably 0.008 to 5 weight %, more preferably 0.01 to 2 weight % based on the total weight of the liquid formulation. The amount of the montelukast should not be less than 0.005 weight % to avoid difficulty in a quality test analysis of a prepared liquid formulation and to resolve the inconvenience of a patient from taking a large amount of the liquid formulation of 40 mL or more, and should not be more than 10 weight % to resolve difficulties in controlling doses where a patient might experience due to the need of taking a small amount of the liquid formulation.

The liquid formulation of the present invention contains montelukast or pharmaceutically acceptable salt thereof as a pharmacologically active ingredient in a solution state, which means that the montelukast or pharmaceutically acceptable salt thereof in the liquid formulation is solubilized and present stably in the liquid formulation in a solution state for a long time.

The “solution state” in the present invention means that the solubility of the montelukast or pharmaceutically acceptable salt thereof measured at room temperature is 95 to 100%.

The liquid formulation of the present invention allows montelukast or pharmaceutically acceptable salt thereof to be present in the formulation in a solution state and shows increased solubility by employing a proper amount of a solubilizer.

The solubilizer in the present invention can be selected from the group consisting of cyclodextrin, polyvinyl caprolactam-polyvinyl acetate-polyethylene glycol graft copolymer, polyoxyethylene derivative and a water-soluble polymer.

The cyclodextrin used as a solubilizer in the present invention can improve water solubility of a substance by water-soluble complexation process. The cyclodextrin is an oligo sugar obtained by applying an enzyme to a starch, which is a monomolecular host which forms an inclusion complex by inserting a guest molecule or atom to a cavity inside a molecule. By such inclusion, the physiochemical property of a guest molecule can be altered resulting in increase of its solubility.

The polyvinyl caprolactam-polyvinyl acetate-polyethylene glycol graft copolymer used as a solubilizer in the present invention is known to increase solubility by changing a molecule into very small particles when mixed with the molecule.

The polyoxyethylene derivative used as a solubilizer in the present invention is a non-ionic surfactant, which can exhibit sufficient solubilizing ability due to its hydrophilic lipophilic balance (HLB) value of between 10 and 16.

The water-soluble polymer used as a solubilizer in the present invention can increase the solubility of a drug by homogeneously dispersing it in a molecular state (amorphous) throughout the whole solid matrix.

In one embodiment of the present invention, the solubilizer is a cyclodextrin, which can be, for example, at least one selected from the group consisting of alpha-cyclodextrin, beta-cyclodextrin, and gamma-cyclodextrin, among which beta-cyclodextrin is preferred.

The cyclodextrin can be employed in an amount of 40 to 200 parts by weight, preferably 60 to 180 parts by weight, and more preferably 80 to 150 parts by weight based on 1 part by weight of montelukast. The solubilized state of a pharmacologically active ingredient in the liquid formulation can be effectively maintained by employing a cyclodextrin in an amount of not less than 40 parts by weight based on 1 part by weight of montelukast, while the increase of related substances in the liquid formulation can be prevented by employing a cyclodextrin in an amount of not more than 200 parts by weight, resulting in improvement in stability of the liquid formulation.

In another embodiment of the present invention, the solubilizer can be polyvinyl caprolactam-polyvinyl acetate-polyethylene glycol graft copolymer, whose average molecular weight can be 90,000 to 140,000 g/mol. Specific examples of such copolymers include Soluplus (BASF), which is currently commercially available.

The polyvinyl caprolactam-polyvinyl acetate-polyethylene glycol graft copolymer can be employed in an amount of 0.8 to 20 parts by weight, preferably 1 to 10 parts by weight, and most preferably 3 to 5 parts by weight based on 1 part by weight of montelukast being added. The solubilized state of the liquid formulation can be effectively maintained by employing the polyvinyl caprolactam-polyvinyl acetate-polyethylene glycol graft copolymer in an amount of not less than 0.8 part by weight based on the amount of montelukast, while the development of poor properties and the increase of related substances can be prevented by employing the copolymer in an amount of not more than 20 parts by weight.

In another embodiment of the present invention, the solubilizer can be a polyoxyethylene derivative, which is, for example, at least one selected from the group consisting of polyoxyethylene castor oil, polyoxyethylene hydrogenated castor oil and polyoxyethylene-polyoxypropylene alkyl ether, preferably, polyoxyethylene hydrogenated castor oil (Cremophor RH 40).

The polyoxyethylene derivative can be employed in an amount of 3 to 15 parts by weight, preferably 5 to 10 parts by weight based on 1 part by weight of montelukast. The solubilized state of the liquid formulation can be effectively maintained by employing the polyoxyethylene derivative in an amount of not less than 3 parts by weight based on the amount of montelukast, while the increase of related substances can be prevented by employing the polyoxyethylene derivative in an amount of not more than 15 parts by weight.

In another embodiment of the present invention, the solubilizer can be a water-soluble polymer, which can be at least one selected from the group consisting of alginic acid, calcium carboxymethyl cellulose, sodium carboxymethyl cellulose, Carbomer, carrageenan, chitosan, guar gum, hypromellose, polyvinyl alcohol, polyethylene oxide, polyvinyl pyrrolidone, hydroxypropyl cellulose, hydroxypropyl ethyl cellulose, vinyl pyrrolidone-vinyl acetate copolymer and Eudragit, among which polyvinyl pyrrolidone is preferred.

The water-soluble polymer can be employed in an amount of 3 to 15 parts by weight, preferably 5 to 10 parts by weight, based on 1 part by weight of montelukast. The solubilized state of the liquid formulation can be effectively maintained by employing the water-soluble polymer in an amount of not less than 3 parts by weight based on the amount of montelukast, while the increase of related substances can be prevented by employing the water-soluble polymer in an amount of not more than 15 parts by weight.

The liquid formulation of the present invention may further comprise a pharmaceutically acceptable additive. The pharmaceutically acceptable additive can be, for example, at least one selected from the group consisting of a sweetening agent, a flavoring agent, a preservative, an emulsifying agent, a coloring agent and a stabilizer (stabilizing agent). By employing a pharmaceutically acceptable additive, the solubilized state of the liquid formulation can be effectively maintained even when it is stored for a long time and medication compliance can be enhanced by improving its taste and flavor.

The sweetening agent improves taste of the liquid formulation, which can employ, for example, at least one substance selected from the group consisting of white sugar, high fructose, sorbitol, mannitol, xylitol, aspartame, steviol glycosides, sucralose, acesulfame potassium and glycerin. The sweetening agent can be employed in an amount of 0.001 to 50 weight %, preferably 0.005 to 30 weight % based on the total weight of the liquid formulation.

The flavoring agent is an additive that makes patients feel a scent when taking the liquid formulation, which can improve the medication compliance. It can employ, for example, at least one substance selected from the group consisting of various fruit flavors such as apple flavor, cherry flavor, orange flavor, strawberry flavor, banana flavor, grape flavor, mango flavor and peach flavor, general fruit flavor, vanilla flavor, mint flavor, and honey flavor. The flavoring agent can be employed in an amount of 0.1 to 5 weight %, preferably 0.2 to 3 weight % based on the total weight of the liquid formulation.

The preservative is an additive that can inhibit the growth of microorganisms during storage of the liquid formulation, which can employ, for example, at least one substance selected from the group consisting of paraoxybenzoates such as paraoxybenzoic acid methyl, paraoxybenzoic acid ethyl, paraoxybenzoic acid propyl and paraoxybenzoic acid butyl; benzoates such as benzoic acid and sodium benzoate; and sorbates such as sorbic acid and potassium sorbate. The preservative can be employed in an amount of 0.01 to 2 weight %, preferably 0.02 to 1 weight % based on the total weight of the liquid formulation.

The stabilizer plays an important role in the maintenance and improvement of the property of the liquid formulation, which can employ, for example, at least one substance selected from the group consisting of meglumine, arginine, lysine, and sodium bicarbonate. The stabilizer can be employed in an amount of 0.001 to 1.0 weight %, preferably 0.005 to 0.5 weight % based on the total weight of the liquid formulation.

The liquid formulation can also employ an emulsifying agent, which can improve the property of the liquid formulation, and a pH adjusting agent, which adjusts the acidity of the liquid formulation to an appropriate level. Additionally, purified water can be employed as a solvent during the preparation of a liquid formulation of the present invention.

As the liquid formulation according to the present invention can maintain its solubility and dissolution, exhibits favorable pharmacological activity over time, and shows favorable stability in terms of decreased production of related substance, it can be utilized for the treatment and prevention of asthma, allergic rhinitis, atopic dermatitis, chronic urticaria, sinusitis, nasal polyps, chronic obstructive pulmonary disease, conjunctivitis including rhino-conjunctivitis, migraine, cystic fibrosis, and viral bronchiolitis, etc.

In addition, the present invention provides a method for preparing a liquid formulation, comprising the steps of:

1) preparing solution 1 by adding to purified water one or more pharmaceutically acceptable additives selected from the group consisting of a sweetening agent, a flavoring agent, a preservative, an emulsifying agent, a coloring agent and a stabilizer, and stirring resulting mixture;

2) preparing solution 2 by adding to purified water a solubilizer selected from the group consisting of cyclodextrin, polyvinyl caprolactam-polyvinyl acetate-polyethylene glycol graft copolymer, polyoxyethylene derivative and a water-soluble polymer, and stirring resulting mixture, and then adding montelukast or pharmaceutically acceptable salt thereof to the mixture, and stirring resulting mixture; and

3) mixing solution 1 with solution 2, and adjusting the concentration of resulting solution by adding purified water thereto.

Preferably, a preparation method of the present invention can comprise the steps of:

1) preparing solution 1 by adding a preservative to purified water and stirring resulting mixture, and then sequentially adding a sweetening agent and a stabilizer to the mixture and stirring resulting mixture;

2) preparing solution 2 by adding to purified water a solubilizer selected from the group consisting of beta-cyclodextrin, polyvinyl caprolactam-polyvinyl acetate-polyethylene glycol graft copolymer, polyoxyethylene derivative and polyvinyl pyrrolidone, and stirring resulting mixture, and then adding montelukast or pharmaceutically acceptable salt thereof to the mixture, and stirring resulting mixture; and

3) adding solution 2 to solution 1, adjusting the concentration of resulting solution by adding purified water thereto, and stirring resulting solution.

According to one embodiment of the present invention, a preparation method of the present invention can comprise the steps of:

i) placing sterile purified water in a preparation tank and heating it to a temperature of 70 to 95° C., preferably about 80° C., and then adding paraoxybenzoic acid methyl and paraoxybenzoic acid propyl to the purified water while stirring resulting mixture;

ii) cooling the solution obtained at step i) to a temperature of 30° C. or lower, preferably 15 to 30° C., and then adding a sweetener thereto, and stirring resulting mixture;

iii) adding to the solution obtained at step ii) a stabilizer, and stirring resulting mixture;

iv) separately, placing sterile purified water in a preparation tank and cooling it to a temperature of 30° C. or lower, preferably 15 to 30° C., and then adding to the purified water a solubilizer selected from the group consisting of beta-cyclodextrin, polyvinyl caprolactam-polyvinyl acetate-polyethylene glycol graft copolymer, polyoxyethylene derivative and polyvinyl pyrrolidone, and stirring resulting mixture;

v) adding montelukast sodium to the solution obtained at step iv), and stirring resulting mixture;

vi) adding the solution obtained at step v) to the solution obtained at step iii), and adding sterile purified water cooled to room temperature thereto to adjust its final volume to 100 mL, and stirring resulting mixture; and

vii) filtrating the final mixture obtained at step vi) with a 1 μm filter, and storing it in a storage tank or an appropriate container.

Hereinafter, the present invention is described more specifically by the following examples, but these are provided only for illustration purposes and the present invention is not limited thereto.

### EXAMPLE 1

### Preparation of Liquid Formulation Containing Montelukast

About 50 mL of sterile purified water was placed in a preparation tank and heated to 80° C., and 0.08 g of paraoxybenzoic acid methyl and 0.02 g of paraoxybenzoic acid propyl were added thereto and dissolved while stirring resulting mixture. After cooling the preparation tank to a temperature of 30° C. or lower, acesulfame potassium was added thereto and stirred, and then 0.01 g of meglumine was added thereto, resulting in solution 1.

Meanwhile, a separate solution was prepared by adding 8.0 g of beta-cyclodextrin to sterile purified water of 25° C., and then adding 0.104 g of montelukast sodium thereto, and stirring the resulting mixture, resulting in solution 2.

Solution 2 was mixed with solution 1, and sterile purified water was added thereto to prepare a solution with a final volume of 100 mL. The prepared final mixture was filtrated with a 1 μm filter, completing of the preparation of a liquid formulation in a solution state.

The prepared liquid formulation showed montelukast solubility of 100%, confirming that montelukast sodium was present in a solution state.

### Examples 2 to 5

### Preparation of Liquid Formulations Containing Montelukast

Other liquid formulations were prepared by the same method as Example 1, except for the composition and amounts described in Table 1.

TABLE 1

| Substances (g) | Ex. 1 | Ex. 2 | Ex. 3 | Ex. 4 | Ex. 5 |
|---|---|---|---|---|---|
| Montelukast sodium | 0.104 | 0.104 | 0.104 | 0.104 | 0.104 |
| (as montelukast) | (0.100) | (0.100) | (0.100) | (0.100) | (0.100) |
| Acesulfame potassium | 0.02 | 0.02 | 0.02 | 0.02 | 0.02 |
| Meglumine | 0.01 | 0.01 | 0.01 | 0.01 | 0.01 |
| Methyl paraoxybenzoate | 0.08 | 0.08 | 0.08 | 0.08 | 0.08 |
| Propyl paraoxybenzoate | 0.02 | 0.02 | 0.02 | 0.02 | 0.02 |
| Beta-cyclodextrin | 8.0 | 15.0 | — | — | — |
| Soluplus | — | — | 0.5 | — | — |
| Cremophor RH 40 | — | — | — | 0.5 | — |
| Polyvinyl pyrrolidone | — | — | — | — | 0.5 |
| Purified Water | aa | aa | aa | aa | aa |
| Total amount | 100 mL | 100 mL | 100 mL | 100 mL | 100 mL |
| Amount of Solubilizer | 80 | 150 | 5 | 5 | 5 |
| (based on 1 part by | | | | | |
| weight of montelukast) | | | | | |

*aa: appropriate amount

### Comparative Examples 1 to 8

### Preparation of Liquid Formulations Containing Montelukast

Comparative liquid formulations were prepared by the same method as Example 1, except for the composition and amounts described in Table 2.

TABLE 2

| | Comp. | Comp. | Comp. | Comp. | Comp. | Comp. | Comp. | Comp. |
|---|---|---|---|---|---|---|---|---|
| Substances (g) | Ex. 1 | Ex. 2 | Ex. 3 | Ex. 4 | Ex. 5 | Ex. 6 | Ex. 7 | Ex. 8 |
| Montelukast | 0.104 | 0.104 | 0.104 | 0.104 | 0.104 | 0.104 | 0.104 | 0.104 |
| sodium | (0.100) | (0.100) | (0.100) | (0.100) | (0.100) | (0.100) | (0.100) | (0.100) |
| (as montelukast) | | | | | | | | |
| Acesulfame | 0.02 | 0.02 | 0.02 | 0.02 | 0.02 | 0.02 | 0.02 | 0.02 |
| potassium | | | | | | | | |
| Meglumine | 0.01 | 0.01 | 0.01 | 0.01 | 0.01 | 0.01 | 0.01 | 0.01 |
| Methyl | 0.08 | 0.08 | 0.08 | 0.08 | 0.08 | 0.08 | 0.08 | 0.08 |
| paraoxybenzoate | | | | | | | | |
| Propyl | 0.02 | 0.02 | 0.02 | 0.02 | 0.02 | 0.02 | 0.02 | 0.02 |
| paraoxybenzoate | | | | | | | | |
| Beta- | — | 3.0 | 21.0 | — | — | — | — | — |
| cyclodextrin | | | | | | | | |
| Soluplus | — | — | — | 2.1 | — | — | — | — |
| Cremophor RH | — | — | — | — | 1.6 | — | — | — |
| 40 | | | | | | | | |
| Polyvinyl | — | — | — | — | — | 1.6 | — | — |
| pyrrolidone | | | | | | | | |
| Poloxamer 407 | — | — | — | — | — | — | 0.5 | — |
| Sorbitan | — | — | — | — | — | — | — | 0.5 |
| mono-oleate 60 | | | | | | | | |
| Purified Water | aa | aa | aa | aa | aa | aa | aa | aa |
| Total amount | 100 mL | 100 mL | 100 mL | 100 mL | 100 mL | 100 mL | 100 mL | 100 mL |
| Amount of | 0 | 30 | 210 | 21 | 16 | 16 | 5 | 5 |
| solubilizer | | | | | | | | |
| (based on 1 | | | | | | | | |
| part by weight | | | | | | | | |
| of montelukast) | | | | | | | | |

*aa: appropriate amount

### Test Example 1

### Observation of the Property of Montelukast Liquid Formulation

The liquid formulations prepared in Examples 1 to 5 and Comparative Examples 1 to 8 were put into transparent glass bottles, and stored at room temperature for 7 days, and 6 months, and were examined for their property and precipitation formation with naked eyes. The results are described in Table 3.

TABLE 3

| | Evaluation of precipitation and property | | | |
|---|---|---|---|---|
| | 7-day storage at room | | 6-month storage at room | |
| | temperature | | temperature | |
| | Precipi- | | Precipi- | |
| | tation | Opacity | tation | Opacity |
| Ex. 1 | − | − | − | − |
| Ex. 2 | − | − | − | − |
| Ex. 3 | − | − | − | − |
| Ex. 4 | − | − | − | − |
| Ex. 5 | − | − | − | − |
| Comp. Ex. 1 | + | − | + | − |
| Comp. Ex. 2 | + | + | + | + |
| Comp. Ex. 3 | − | − | − | − |
| Comp. Ex. 4 | − | + | + | + |
| Comp. Ex. 5 | − | − | − | − |
| Comp. Ex. 6 | − | − | − | − |
| Comp. Ex. 7 | − | + | + | + |
| Comp. Ex. 8 | − | + | + | + |

− : no precipitation, transparent,
+ : precipitation, opaque

As shown in Table 3, the liquid formulations of Examples 1 to 5 of the present invention were transparent without formation of any precipitation for 6 months at room temperature. On the other hand, Comparative Example 1, in which no solubilizer was added, also was transparent, but recrystallization of montelukast sodium occurred and precipitation was formed at the bottom. In addition, like Comparative Example 2, when the amount of beta-cyclodextrin was insufficient, the solubilizing ability for the main ingredient was decreased resulting in opacity and precipitation formation. In the case of Comparative Example 4, the solubilizing ability for the main ingredient was sufficient, but due to the low solubility of Soluplus in water, it became opaque. In Comparative Examples 7 to 8 in which other solubilizers were added, the formulations became opaque on day 7 at room temperature, and yellow precipitations were observed at the end of the test at room temperature.

### TEST EXAMPLE 2

### Evaluation of the Solubility of Montelukast Liquid Formulation

After 2 to 3 days from the preparation, the liquid formulations prepared in Examples 1 to 5 and Comparative Examples 1 to 8 were centrifuged at 16,000 rpm for 20 minutes, and the supernatants were filtrated with 0.45 μm filters, and then methanol was added to dilute them to 1/4, and then the solubility was analyzed according to the following analysis conditions.

<Condition for Analysis of Montelukast Solubility>

Instrument: HPLC (Hitachi 2000 series, Japan)

Detector: ultraviolet absorption spectrophotometer (wavelength: 225 nm)

Column: stainless steel column (inner diameter: about 4.6 mm; length: about 15 cm) packed with octylsilyl silica gel (particle diameter: 5 μm) for liquid chromatography (Inertsil C8, GL Science Co.)

Mobile phase: 0.025 mol/L potassium dihydrogen phosphate solution (adjusted to a pH of 6.6 with 10 mol/L sodium hydroxide solution):acetonitrile=4:6 (v/v)

Flow rate: 1.0 mL/min.

Column temperature: 45° C.

Results of solubility test of montelukast liquid formulations measured under the above analysis conditions are described in Tables 4 and 5.

TABLE 4

| | Ex. 1 | Ex. 2 | Ex. 3 | Ex. 4 | Ex. 5 |
|---|---|---|---|---|---|
| Input of | 1,000 | 1,000 | 1,000 | 1,000 | 1,000 |
| montelukast | | | | | |
| (μg/mL) | | | | | |
| Solubility of | 999.8 | 1,000 | 995.7 | 999.1 | 998.4 |
| montelukast | | | | | |
| (μg/mL) | | | | | |
| Solubility | 100.0% | 100.0% | 99.6% | 99.9% | 99.8% |
| per input | | | | | |
| (w/w %) | | | | | |

TABLE 5

| | Comp. | Comp. | Comp. | Comp. | Comp. | Comp. | Comp. | Comp. |
|---|---|---|---|---|---|---|---|---|
| | Ex. 1 | Ex. 2 | Ex. 3 | Ex. 4 | Ex. 5 | Ex. 6 | Ex. 7 | Ex. 8 |
| Input of | 1,000 | 1,000 | 1,000 | 1,000 | 1,000 | 1,000 | 1,000 | 1,000 |
| montelukast | | | | | | | | |
| (μg/mL) | | | | | | | | |
| Solubility of | 100.3 | 357.2 | 1,000 | 991.7 | 986.9 | 994.3 | 479.6 | 523.4 |
| montelukast | | | | | | | | |
| (μg/mL) | | | | | | | | |
| Solubility per | 10.3% | 35.7% | 100.0% | 99.2% | 98.7% | 99.4% | 48.0% | 52.3% |
| input (w/w %) | | | | | | | | |

In the liquid formulations of Examples 1 to 4 and Comparative Examples 1 to 8, montelukast sodium was added at a concentration of 1,040 μg/mL, which corresponds to a concentration of 1,000 μg/mL of montelukast, assuming that the entire amount of inputted montelukast has been dissolved. The results confirm that the liquid formulations prepared in Examples 1 to 5 and Comparative Examples 3 to 6, showed solubility of 98 to 100 w/w % per input, while the liquid formulations prepared in Comparative Examples 1, 2, 7 and 8 had insufficient solubilizing ability and were not able to completely solubilize montelukast.

### TEST EXAMPLE 3

### Dissolution Test of Montelukast Liquid Formulation

The liquid formulations prepared in Examples 1 to 5 and Comparative Examples 1 to 8 were stored for 7 days at room temperature, and dissolution test was conducted in accordance with the dissolution test method under the United States Pharmacopeia (USP).

Specifically, 900 mL of 0.5% sodium lauryl sulfate solution was used as an eluent, and paddle method was used. The temperature of the eluent was 37±5° C., and the stirring rate was 50 rpm. 5, 10, 15, 20, 30, 45 and 60 minutes after the dissolution test, 3 mL samples were taken and equal amount of the eluent was added thereto. Then, the solutions acquired at the dissolution test were filtrated with 0.45 μm membrane filter, and dissolution rate of montelukast was analyzed by liquid chromatography of the following conditions.

<Conditions for Dissolution Test Analysis of Montelukast>

Instrument: HPLC (Hitachi 2000 series, Japan)

Detector: ultraviolet absorption spectrophotometer (wavelength: 389 nm)

Column: stainless steel column (inner diameter: about 3 mm; length: about 10 cm) packed with phenyl-silica gel (particle diameter: 5 μm) for liquid chromatography (Inertsil Phenyl, 10 cm×3.0 mm, 5 μm)

Mobile phase: a solution obtained by adding 500 mL of 0.2% (v/v) trifluoroacetic acid aqueous solution to 500 mL of 0.2% (v/v) trifluoroacetic acid acetonitrile solution.

Flow rate: 0.9 mL/min.

Column temperature: 50° C.

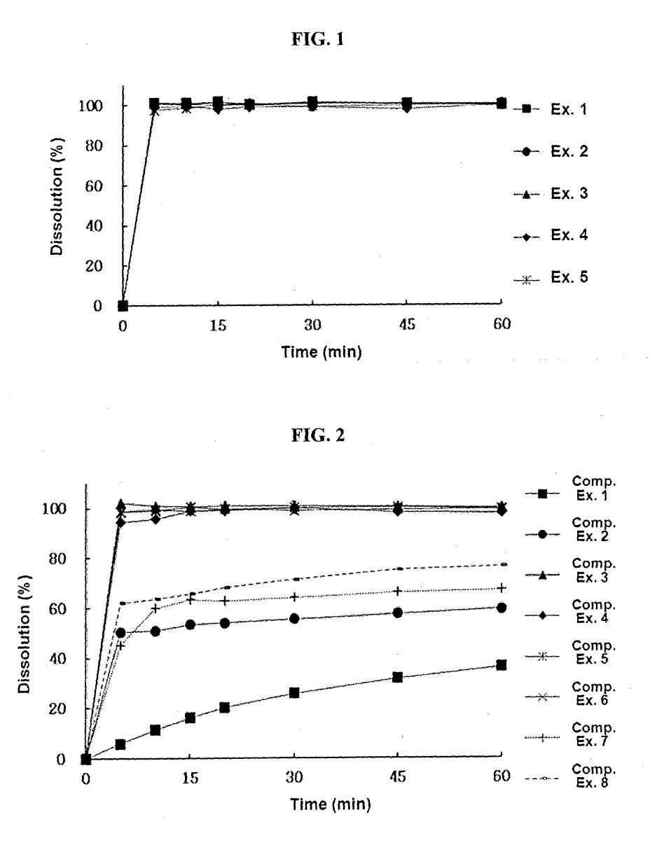

The dissolution test results of the montelukast liquid formulations measured under the above analysis conditions are shown in Tables 6 and 7, and FIGS. 1 and 2.

TABLE 6

| | | Ex. 1 | Ex. 2 | Ex. 3 | Ex. 4 | Ex. 5 |
|---|---|---|---|---|---|---|
| Initial | | 0% | 0% | 0% | 0% | 0% |
| 5 | minutes | 101.0% | 100.4% | 101.1% | 98.9% | 97.5% |
| 10 | minutes | 100.8% | 100.2% | 100.5% | 99.4% | 98.4% |
| 15 | minutes | 101.6% | 100.2% | 101.0% | 98.0% | 99.8% |
| 20 | minutes | 100.3% | 100.4% | 100.8% | 99.1% | 100.3% |
| 30 | minutes | 101.7% | 100.8% | 101.2% | 99.2% | 99.6% |
| 45 | minutes | 100.5% | 100.7% | 100.6% | 98.0% | 99.8% |
| 60 | minutes | 100.0% | 100.6% | 100.2% | 99.8% | 100.2% |

TABLE 7

| | Comp. | Comp. | Comp. | Comp. | Comp. | Comp. | Comp. | Comp. |
|---|---|---|---|---|---|---|---|---|
| | Ex. 1 | Ex. 2 | Ex. 3 | Ex. 4 | Ex. 5 | Ex. 6 | Ex. 7 | Ex. 8 |
| Initial | 0% | 0% | 0% | 0% | 0% | 0% | 0% | 0% |
| 5 minutes | 5.9% | 50.4% | 102.1% | 94.4% | 98.4% | 98.6% | 45.2% | 62.0% |
| 10 minutes | 11.5% | 51.0% | 100.7% | 95.7% | 98.9% | 99.3% | 59.9% | 63.5% |
| 15 minutes | 16.2% | 53.5% | 100.6% | 98.7% | 100.3% | 98.8% | 63.3% | 65.6% |
| 20 minutes | 20.2% | 54.0% | 100.9% | 98.9% | 99.6% | 99.5% | 62.7% | 68.2% |
| 30 minutes | 25.8% | 55.5% | 100.7% | 99.9% | 100.0% | 98.9% | 64.2% | 71.2% |
| 45 minutes | 31.7% | 57.6% | 100.5% | 98.2% | 100.2% | 99.3% | 66.3% | 75.2% |
| 60 minutes | 36.5% | 59.5% | 100.0% | 98.0% | 99.5% | 99.5% | 67.3% | 76.6% |

As shown in Table 6 and FIG. 1, liquid formulations of Examples 1 to 5 showed favorable dissolution rates. However, as shown in Table 7 and FIG. 2, Comparative Example 1, to which no solubilizer was added, showed low dissolution rate with recrystallization of montelukast, and Comparative Example 2, in which solubilizing ability of beta-cyclodextrin was low, showed low dissolution rate with precipitation of a certain amount of the main ingredient.

### TEST EXAMPLE 4

### Analysis of Related Substances of Montelukast Liquid Formulation

After placing the liquid formulations of Examples 1 to 5 and Comparative Examples 1 to 8 in a light-shielding closed glass container and storing it under accelerated conditions, the amount of production of montelukast sulfoxide and total related substances of montelukast was analyzed in accordance with the following conditions for analyzing related substances described below.

<Accelerated Storage Conditions>

Storage conditions: 40° C., relative humidity of 75%, light-shielding closed glass containers

Test timing: initial, 1 month, 3 months and 6 months

Objects to be analyzed: montelukast related substances

<Conditions for Analyzing Montelukast Related Substances>

Instrument: HPLC (Hitachi 2000 series, Japan)

Detector: ultraviolet absorption spectrophotometer (wavelength: 238 nm)

Column: stainless steel column (inner diameter of about 4.6 mm, a length of about 25 cm) packed with diisopropyl phenethyl silica gel (particle diameter of 5 μm) for liquid chromatography (Zorbax SB-Phenyl, Agilent)

Mobile phases:

A—purified water containing 0.1% (v/v) trifluoroacetic acid

B—acetonitrile solution containing 0.1% (v/v) trifluoroacetic acid

Dissolution conditions for mobile phase

| Time (min) | A (%) | B (%) |
|---|---|---|
| 0 | 60 | 40 |
| 20 | 10 | 90 |
| 30 | 10 | 90 |
| 31 | 60 | 40 |
| 35 | 60 | 40 |

Flow rate: 1.5 mL/min.

Column temperature: 25° C.

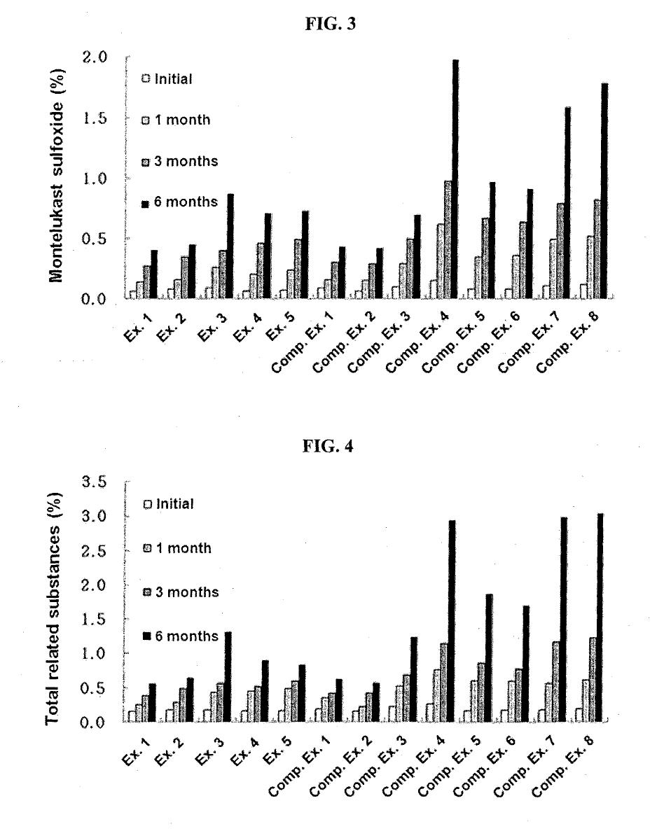

The amount of produced montelukast sulfoxide and total related substances measured under the above analysis conditions are shown in Table 8, and FIGS. 3 and 4.

TABLE 8

| | Initial | | 1 month | | 3 months | | 6 months | |
|---|---|---|---|---|---|---|---|---|
| | | Total | | Total | | Total | | Total |
| | Sulf- | related | Sulf- | related | Sulf- | related | Sulf- | related |
| Sample | oxide | substances | oxide | substances | oxide | substances | oxide | substances |
| Ex. 1 | 0.06% | 0.15% | 0.14% | 0.25% | 0.27% | 0.38% | 0.40% | 0.56% |
| Ex. 2 | 0.08% | 0.17% | 0.16% | 0.29% | 0.35% | 0.49% | 0.45% | 0.65% |
| Ex. 3 | 0.09% | 0.17% | 0.26% | 0.43% | 0.40% | 0.56% | 0.87% | 1.18% |
| Ex. 4 | 0.06% | 0.16% | 0.20% | 0.45% | 0.46% | 0.52% | 0.71% | 0.90% |
| Ex. 5 | 0.07% | 0.16% | 0.24% | 0.49% | 0.49% | 0.59% | 0.73% | 0.83% |
| Comp. Ex. 1 | 0.09% | 0.19% | 0.16% | 0.35% | 0.30% | 0.42% | 0.43% | 0.62% |
| Comp. Ex. 2 | 0.06% | 0.15% | 0.15% | 0.22% | 0.29% | 0.42% | 0.42% | 0.57% |
| Comp. Ex. 3 | 0.10% | 0.22% | 0.29% | 0.52% | 0.50% | 0.68% | 0.70% | 1.29% |
| Comp. Ex. 4 | 0.15% | 0.26% | 0.62% | 0.76% | 0.98% | 1.15% | 1.98% | 2.94% |
| Comp. Ex. 5 | 0.08% | 0.16% | 0.35% | 0.60% | 0.67% | 0.86% | 0.97% | 1.87% |
| Comp. Ex. 6 | 0.08% | 0.17% | 0.36% | 0.59% | 0.64% | 0.77% | 0.91% | 1.69% |
| Comp. Ex. 7 | 0.11% | 0.18% | 0.49% | 0.56% | 0.79% | 1.17% | 1.59% | 2.99% |
| Comp. Ex. 8 | 0.12% | 0.19% | 0.52% | 0.61% | 0.82% | 1.23% | 1.79% | 3.04% |

These results indicate that, in Examples 1 to 5 and Comparative Examples 1 to 8, the lower the proportion of beta-cyclodextrin in the solution is, the better the stability of the solution is obtained, and as the proportions of Soluplus, Cremophor RH 40 and polyvinyl pyrrolidone in the solution increase, the amount of produced montelukast sulfoxide and related substances increases. In addition, the total production of related substances in Comparative Examples 3 to 8 exceeded the standard limit of 1.2%, and, thus, the liquid formulations of Comparative Examples 3 to 8 showed inferior stability as compared to the liquid formulations of the present invention.

What is claimed is:

1. A liquid formulation comprising:
a solubilizer selected from the group consisting of cyclodextrin, polyvinyl caprolactam-polyvinyl acetate-polyethylene glycol graft copolymer, polyoxyethylene derivative and a water-soluble polymer; and
montelukast or pharmaceutically acceptable salt thereof in a solution state as an active ingredient.

2. The liquid formulation of claim 1, wherein the montelukast or the pharmaceutically acceptable salt thereof is employed in an amount of 0.005 to 10 weight % based on the total weight of the liquid formulation.

3. The liquid formulation of claim 1, wherein the solubilizer is cyclodextrin and is employed in an amount of 40 to 200 parts by weight based on 1 part by weight of montelukast.

4. The liquid formulation of claim 1, wherein the solubilizer is polyvinyl caprolactam-polyvinyl acetate-polyethylene glycol graft copolymer and is employed in an amount of 0.8 to 20 parts by weight based on 1 part by weight of montelukast.

5. The liquid formulation of claim 1, wherein the solubilizer is polyoxyethylene derivative and is employed in an amount of 3 to 15 parts by weight based on 1 part by weight of montelukast.

6. The liquid formulation of claim 1, wherein the solubilizer is a water-soluble polymer and is employed in an amount of 3 to 15 parts by weight based on 1 part by weight of montelukast.

7. The liquid formulation of claim 1, wherein solubility of the montelukast or the pharmaceutically acceptable salt thereof measured at room temperature is 95 to 100%.

8. The liquid formulation of claim 1, further comprising one or more pharmaceutically acceptable additives selected from the group consisting of a sweetening agent, a flavoring agent, a preservative, an emulsifying agent, a coloring agent and a stabilizer.

9. The liquid formulation of claim 1, wherein the cyclodextrin is at least one selected from the group consisting of alpha-cyclodextrin, beta-cyclodextrin, and gamma-cyclodextrin.

10. The liquid formulation of claim 1, wherein the polyoxyethylene derivative is at least one selected from the group consisting of polyoxyethylene castor oil, polyoxyethylene hydrogenated castor oil and polyoxyethylene-polyoxypropylene alkyl ether.

11. The liquid formulation of claim 1, wherein the water-soluble polymer is at least one selected from the group consisting of alginic acid, calcium carboxymethyl cellulose, sodium carboxymethyl cellulose, Carbomer, carrageenan, chitosan, guar gum, hypromellose, polyvinyl alcohol, polyethylene oxide, polyvinyl pyrrolidone, hydroxypropyl cellulose, hydroxypropyl ethyl cellulose, vinyl pyrrolidone-vinyl acetate copolymer and Eudragit.

12. A method for preparing the liquid formulation of claim 1, comprising the steps of:
1) preparing solution 1 by adding to purified water one or more pharmaceutically acceptable additives selected from the group consisting of a sweetening agent, a flavoring agent, a preservative, an emulsifying agent, a coloring agent and a stabilizer, and stirring resulting mixture;
2) preparing solution 2 by adding to purified water a solubilizer selected from the group consisting of cyclodextrin, polyvinyl caprolactam-polyvinyl acetate-polyethylene glycol graft copolymer, polyoxyethylene derivative and a water-soluble polymer, and stirring resulting mixture, and then adding montelukast or pharmaceutically acceptable salt thereof to the mixture and stirring resulting mixture; and
3) mixing solution 1 with solution 2 and adjusting the concentration of resulting solution by adding purified water thereto.
